(19) United States
(12) Patent Application Publication
(10) Pub. No.: US 2016/0206673 A1
(43) Pub. Date: Jul. 21, 2016

(54) Composition and Method for Improving Sexual Performance

(71) Applicants: David Miller, Upper Marlboro, MD (US); Stephanie Lynn, Upper Marlboro, MD (US)

(72) Inventors: David Miller, Upper Marlboro, MD (US); Stephanie Lynn, Upper Marlboro, MD (US)

(21) Appl. No.: 14/598,033

(22) Filed: Jan. 15, 2015

(51) Int. Cl. A61K 36/258; A61K 36/537; A61K 49/00; A61K 36/90; A61K 36/73; A61K 36/898; A61K 36/23; A61K 36/888; A61K 36/71; A61K 36/533; A61K 36/288; A61K 36/899; A61K 36/287; A61K 36/53; A61K 36/185; A61K 36/74; A61K 36/82; A61K 36/88; A61K 9/00

(52) U.S. Cl. CPC A61K 36/258; A61K 9/0095; A61K 9/0053; A61K 36/537; A61K 49/0004; A61K 36/90; A61K 36/73; A61K 36/898; A61K 36/23; A61K 36/888; A61K 36/71; A61K 36/533; A61K 36/288; A61K 36/899; A61K 36/287; A61K 36/53; A61K 36/185; A61K 36/74; A61K 36/82; A61K 36/88; A23L 1/3002; A23L 1/30; A23V 2002/00

(57) ABSTRACT

The present application relates to a synergistic blend of ingredients and a method of using the synergistic blend for benefiting health and improving sexual performance.

### BACKGROUND OF THE INVENTION

1. Field of the Invention

The present invention relates to methods and compositions for providing multiple health benefits and for positively improving sexual potency in a living subject. The present invention further relates to compositions for improving quality, strength and duration of penile erections for prostate surgery candidate before and after a prostate procedure.

2. Discussion of the Background Art

Millions of people all over the earth experience impotence and other problems associated with sexual response. The term impotence is used to describe a condition occurring primarily in males and generally characterized by an inability to obtain and maintain a penile erection. In men the condition is also sometimes referred to as erectile dysfunction.

In the context of the present invention, the term “erectile dysfunction” refers to certain disorders of the cavernous tissue of the penis and the associated fascia. These disorders result in impotence. Impotence is estimated to affect about more than ten million men in the United States alone. Impotence can result from disruption of any of numerous physiological or psychological factors which cause the blood flow to and from the penis to remain in balance thereby preventing retention of sufficient blood to cause rigid dilation of the corpus cavernosa and spongiosa. In the context of the present invention, the term “impotence” is used in its broadest sense as the inability to attain a sexually functional erection when desired.

Of considerable concern is impotency in men which is often associated with prostate surgery or treatment related to prostate problems. Regardless of whether the nerves were spared during surgery or whether the most precise dose planning was used during radiation therapy, nearly all men will experience some erectile dysfunction for the first few months after treatment. The reason for this is that nerves and blood vessels that control the physical aspect of an erection are incredibly delicate, and any trauma (for example, surgery) to the area will result in changes to the natural order.

It is estimated that within one year after surgery or treatment, about fifty to sixty percent of men who undergo nerve-sparing prostatectomy will still not have returned to their pre-treatment function. At about two years, about thirty to sixty percent will not have returned to pre-treatment function. These rates vary widely depending on the surgeon and level of experience, and how outcomes are reported (i.e., whether a study measures a doctor's opinion of a patient's sexual function or whether the survey actually asks the patient about this side effect).

For men who have undergone radiation therapy as opposed to the removal of the entire prostate, the numbers are slightly better, but tend not to improve too much over time. Men who undergo procedures that are not designed to minimize side effects and/or those whose treatments are administered by physicians who are not proficient in the procedures will fare worse. In addition, men with other diseases or disorders that impair their ability to maintain an erection (such as diabetes or vascular problems) will have a more difficult time returning to pre-treatment function.

When a man is sexually aroused, the erectile nerves running alongside the penis stimulate the muscles to relax, allowing blood to rush in. At the same time, tiny valves at the base of the penis lock shut, preventing the blood from flowing back out and therefore causing the penis to stay rigid. An amount of loss of function in this sequence will result in erectile dysfunction or impotence.

As a solution to the problem of erectile dysfunction, there have been developed oral medications (sildenafil (Viagra), tadalafil (Cialis), and vardenafil (Levitra)) which relax the muscles in the penis, allowing blood to rapidly flow in. On average, the drugs take about an hour to begin working. The erection helping effects of sildenafil and vardenafil last for about up to eight hours and tadalafil about 36 hours. About seventy five percent of men who undergo nerve-sparing prostatectomy or more precise forms of radiation therapy have reported successfully achieving erections after using these drugs. Results of use of these drugs by men who have had less nerves spared during surgery is not as encouraging.

Further, these drugs are not for everyone. Many men with angina or other heart problems take medications that contain nitrates to help the blood flow better to the heart. All three agents used for erectile dysfunction can affect the way that the nitrates work and cause blood pressure to drop to dangerously low levels. They can also interfere with alpha-blockers, drugs that are commonly used in men with the noncancerous growth of the prostate known as BPH, and that are often used in men following certain types of radiotherapy, particularly brachytherapy.

In addition to the oral medications, there are a number of alternative treatments that have been reported to be helpful to men with erectile dysfunction. For example, MUSE is a medicated pellet about half the size of a grain of rice that is inserted into the urethra through the opening at the tip of the penis using a disposable plastic applicator. Like the oral medications, it too stimulates blood flow into the penis. With MUSE, an erection typically occurs within ten minutes after insertion of the pellet and can last for thirty to sixty minutes. About forty percent of men have reported successfully achieving erections after using this drug, but the results are often inconsistent.

Another remedy is Caverject. It uses the same drug that is in the MUSE pellets, but delivers it via an injection directly into the penis. It takes about ten minutes to work and lasts for about thirty minutes. Although nearly ninety percent of men using Caverject reported erections about six months after therapy, most men are not willing to inject themselves regularly. Therefore the treatment is not often used for long periods of time.

Because some men are unwilling or unable to use any form of medication to help improve erectile function, there are mechanical devices available. The vacuum constriction device (or vacuum pump) creates an erection mechanically by forcing blood into the penis using a vacuum seal. Because the blood starts to flow back out once the vacuum seal is broken, a rubber ring is rolled onto the base of the penis, constricting it sufficiently so that the blood does not escape. About eighty percent of men find this device successful. However, it also has a high drop-out rate. One drawback is that the constriction ring at the base of the penis is effectively cutting off fresh circulation. Because of this effect, it is crucial that the ring be removed immediately after intercourse, or the tissue can be damaged due to lack of flowing oxygen.

Another option for treating erectile dysfunction is the surgically inserted penile implant. With the three-piece implant, a narrow flexible plastic tube is inserted along the length of the penis, a small balloon-like structure filled with fluid is attached to the abdominal wall and a release button is inserted into the testicle. The penis remains flaccid until an erection is desired, at which point the release button is pressed and fluid from the balloon rushes into the plastic tube. As the tube straightens from being filled with the fluid, it pulls the penis up with it, creating an erection. This method is generally effective, but is not desirable as a first choice for most men.

Various sources of evidence show that dehydroepiandrosterone (DHEA) increases libido in women and helps erectile dysfunction in men. DHEA appears to be safe at low doses. However, it has been known to cause acne. Also, some evidence shows that taking high doses of L-arginine improves erectile dysfunction by stimulating blood vessels to open wider for improved blood flow. A drawback is that side effects may include nausea, cramps and diarrhea. Further it can have a negative effect if taken with other vasodilating agents such as Viagra.

There are studies showing that various forms of ginseng, including various forms and types of Panax ginseng improved sexual function in men with erectile dysfunction. Ginseng contains many active ingredients. It appears to be safe used on a short-term basis but long term use may produce side effects. Insomnia is a common side effect.

Yohimbine is a component of the bark from an African tree. A number of clinical trials have shown that it can improve sexual dysfunction associated with a drug used to treat depression. This herb has been linked to a number of side effects, including increased blood pressure, fast or irregular heartbeat, and anxiety. Use of Yohimbine without a doctor's supervision can create serious health risks.

Ginkgo Biloba has potential to increase blood flow to the penis. However, there is scarce or no evidence of benefit for erectile dysfunction. A negative side effect is that Ginkgo may increase the risk of bleeding.

Finally, there exists an almost inexhaustible list of substances such as herbs, spices and other components that are believed to provide various health benefits. Many of such health benefits are improvement in virility, libido and sexual performance and desire in males and females. The benefits of these substances sometimes vary widely from culture to culture. They range from common household herbs and spices to extremely exotic and rare roots, barks, fruits and leaves and various extracts and components thereof. Some of these come with demonstrated serious negative side effects and adverse effects on health of those who use them. Some side effects are associated with quantity of use, length of time used, a particular medical condition or disposition of the individual using them, use in combination with one or more other substances, method of use, frequency of use and state of potency or deterioration in freshness. Many of the substances may be effective for one individual and ineffective for another.

Other drawbacks to these substances is that a potential user may be discouraged by taste, color, consistency, difficulty of preparation, form of delivery (pill, powder, liquid, solid, gas, paste, patch, for example). In other words, an individual may simply have an aversion to taking pills (for example) and an individual may simply not like the taste (for example) and an individual may be discouraged by the extra effort that it takes to remember to take the substance.

Another drawback to the methods and compositions of the background art is frustration with the method of delivery. For example, if a pill or mixture is administered (including self-administered) for a desired result, it may become a tedious task. A user may forget to take the various combinations or just merely become frustrated with having to be administered these in addition to intake of other food or drink items. Currently, effective vehicles for easing the frustration with administration of available products are rare or do not exist.

Also, while many herbs and other components are purported to have certain properties, there is uncertainty which active ingredients are effective. While it is common for herbs and other ingredients to be subject to various amounts of liquids and heats to create a tincture or liquid extract, there is a general lack of controls to ensure that the vital components are preserved through the process. In other words, combinations of herbs and other ingredients are mixed together, and it is assumed that the important constituents will survive extraction process. In reality however, heat can actually destroy important constituents.

Further, reactions between constituents can cause alterations which reduce the potency of the product. In many herbs or folk medicines, they are believed to yield certain positive results, but the component or ingredient in the herb is often unknown. Even in more modern production facilities quantification and controls at various steps of the process are nonexistent. This is sometimes due to lack of sophistication or equipment necessary to implement these controls. Even if various types of test instruments such as process gas chromatographs or mass spectrometers were available, the active ingredients which yield the desired results may be unknown. As a result, many conclusions are reached with little scientific data to back them up.

Still further, methods of satisfactorily quantifying amounts of components in various formulations are very limited, nonexistent or ineffective. In this respect, there is needed a method to ensure the quality and efficacy of the product during the manufacturing process.

Further, there is a need for a product and method for providing a health benefit and improving sexual performance in an individual that overcomes the limitations and disadvantages of the background art.

### SUMMARY OF THE INVENTION

Accordingly, the present invention is directed to one or more compositions and methods of manufacturing and using compositions to provide health benefits and improvements in sexual performance that substantially obviate one or more problems due to limitations and disadvantages of the background art.

Additional advantages, objects, and features of the invention will be set forth in part in the description which follows and in part will become apparent to those having ordinary skill in the art upon examination of the following or may be learned from practice of the invention. The objectives and other advantages of the invention may be realized and attained by the features particularly pointed out in the written description hereof.

To achieve these objects and other advantages and in accordance with the purpose of the invention, as embodied and broadly described herein, there is provided a composition for providing a health benefit to a human comprising a synergistic blend for benefiting health and improving sexual performance in an animal comprising one or more components selected from among horny goat weed and pomegranate seeds and dandelion and one or more components selected from among a group comprising various forms of ginseng, the forms including asian ginseng, siberian ginseng, red white Ginseng and american ginseng and one or more components selected from among ginger root, carob bean, lady's mantle and cayenne pepper and one or more components selected from among peanuts, extract of watermelon rind, l-arginine, chaste tree berry and nitric oxide and one or more components selected from among black cohosh, guarana and kola nut and one or more components selected from among oatstraw and horsetail and one or more components selected from among damiana leaf and red raspberry and one or more components selected from among sarsaparilla root and sassafras root and one or more components selected from among flax seeds and saw palmetto and one or more components selected from among maca root, suma, parsley and muira puama and one or more components selected from among tribulus terrestres kelp pedalium murex and DHEA and one ore more components selected from among cnidium momier, tonka bean and vanilla grass and wherein the blend comprises one or more of herbal and non herbal extracts and components, wherein the blend is infused into one or more of a solid and semi-solid medium, said medium including one or more of a coffee bean and a part thereof, said coffee bean and part thereof being one or more of caffeinated and non-caffeinated, and wherein said blend is administered as one or more of a liquid, a solid and a semi solid, and wherein an administration of said blend to a consumer provides one or more health benefits to a consumer, the health benefits including maintaining a penile erection for a duration of time, and a synergistic blend for benefiting health and improving sexual performance in an animal comprising one or more components selected from among gota and sage and one or more components selected from lady's mantle and gingko biloba and one or more components selected from among crane's bill and sencha fuji and one or more components selected from among cabbage and kola nut and one or more components selected from among periwinkle and rosemary and one or more components selected from among yarrow and holy basil and one or more components selected from among shepherd's purse and lavender and one or more components selected from among black haw and chamomile and one or more components selected from among peppermint and spearmint and wherein the blend comprises one or more of herbal and non herbal extracts and components, wherein the blend is infused into one or more of a solid and semi-solid medium, said medium including one or more of a coffee bean and a part thereof, said coffee bean and part thereof being one or more of caffeinated and non-caffeinated, and wherein said blend is administered as one or more of a liquid, a solid and a semi solid, and wherein an administration of said blend to a consumer provides one or more health benefits to a consumer thereof, the health benefits including maintaining a penile erection for a duration of time and wherein the blend is administered in combination with a favorite food or beverage and wherein the blend is administered in combination with a favorite food or beverage and wherein additional ingredients having a benefit selected from among taste, smell, color, synergy and texture are selected from among one or more of abalone shell, abizzia bark, abuta, acanthopanax. aconite, agastache, agrimony, akebia, alfalfa, alisma, aloe vera, amalaki, amasake, american ginseng, amla, andrographis paniculata, anemarrhena, angelica, angelica du huo, anise, aniseed, annato, apricot seed, aquilaria, aralia, arisaema flower, arnica, artemisia annua, artemsisia annua, artist conk, asafetida, asafoetida, ashwagandha, asparagus fern root, asparagus root, asparagus shoots, astragalus, astralagus, atractylodes, korean, siberian or other ginseng, pueraria lobata root, gaba, wild yam extract, muira puama extract, HCl, vitamin b6, eleutherococcus extract, xanthroparmelia scarbrosa, velvet deer antler, pumpkin seed, stinging nettle, astragalus, licorice extract, ho shou wu extract, hops, zinc, and boron; saffron, tumeia aphrodisiaca, irish moss, and fake unicorn root, panax ginseng, red ginseng, rasberry, red raspberry, curculigo, mucuna pruiens, ostrea gigas, pyrola calliantha, cuscuta chinensis, dong quai, cynomorium songaricum, bitter melon, momordica, american ginseng, bilberry, vitamin E, DHEA, balm of gilead, baptisia, barberry, bay leaf, bayberry, begonia, beleric myrobalan, benzoin, bilberry, biota leaves, biota seeds, bitter melon, bitter melon juice, bitter orange, black atractylodes, black cumin, black currant seed oil, black current, black nightshade, black sesame seed, black sesame seeds, black soybean, blackberry lily, bladderwrack, blessed thistle, bletilla, bloodroot, blue cohosh, blue flag, blue vervain, bogbean, boldo, bolete, boneset, borage, borneol, boswellia, bovine tracheal cartilage, brahmi, buchu, buckthorn, buddleai flower, bugleweed, buglweed, bupleurum, bupleurum root, burdock, burdock root, butcher's broom, butcher's-broom, cajueiro fruit, cajuput, calamus, calendula, california poppy, camellia, camomile, caraway, cardamom, cascara sagrada, cassava, cassia seed, castor oil plant, castor-oil plant, catha, catnip, cat's claw, cattail pollen, cayenne pepper, ceanothus, celandine, celeriac, cernilton, cernitin, chanca piedo, chaparral, chebulic myrobalan, chelidonium, chelone, chenopodium, cherry stems, chickweed, chicory, chinaberry bark, chinese angelica, chinese quince, chinese raspberries, chinese thoroughwax, chinese water melon, chrysanthemum, chuchuasi, cibotium, cijuwa, cimicifuga, cinnabar, cinnamon, cinnamon twig, cistanche, citrus peel, cleavers, clematis root, cocklebur fruit, codonopsis, coffee, coix, collinsonia, coltsfoot, coltsfoot flower, comfrey, coptis, cordyceps, corn silk, cornsilk, corydalis, cow bezoar, cramp bark, cranesbill root, cress, culver's root, cumin, curculigo, curcumin, cuscutu cheninsis, cuttlefish bone, cyperus, cynomorium songaricon, cypress, dahlia, day lily, deer antler, deglycyrrhizinated licorice, dendrobium orchid, devil's claw, DHEA, devils' club, dill, dill weed, dioscorea, dipsacus root, dogwood berries, don sen, dong quai, drynaria, echinacea, eclipta, elder, elder bark, elderberry, elderberry extract, elderflower, elecampane, eleuthero, emblic myrobalan, enoki, epazote, ephedra, epimedium, epimedium leaf, eucalyptus, eucommia bark, euphorbia, ansui, euryale seeds, evening primrose, eyebright, false unicorn, fennel, fennel seed, fenugreek, feverfew, fleeceflower, fleeceflower, stem, florence fennel, forsythia, forsythia fruit, fritillaria, fo-ti, fu ling, galangal, garcinia, gardenia fruit, garlic, garlic chives, gastrodia, gentian, gentian root, geranium, ginkgo nut, ginseng, glehnia root, golden seal, goldenrod, goldenseal, goti berries, gota kola, gotu kola, grapefruit seed extract, gravel root, green tangerine peel, green tea, green tea extract, grindelia, ground ivy, guaiacum, guarana, guggul, gugl, gugulipid, gymnema sylvestre, gypsum, hato mugi, hawthorn berries, hawthorn berry, hemp seed, hercampure, hermatitum, hibiscus, ho shou wu, honeysuckle, honeysuckle flowers, hops, horehound, horse chestnut, horse, hestnuts, horseradish, horsetail, houttuynia, huckleberries, huitlacoche, iceland moss, immature bitter orange, impatiens, imperata, inula flower, ipecacuanha, isatis root, japanese elderberry, japanese knotweed, japanese turf lily, jasmine, jatoba tree, jinengo, jujube date, juniper berries, kava kava, khella, knotweed, kudzu, kuzu root, lady's slipper, lantana, large-leafed gentiana, laver, ledebouriella, lemon balm, lemon grass, licorice root, ligusticum, ligustrum berries, ligustrun, lily bulb, linden flowers, lindera root, litchi, lo han kuo, lobelia, lomatium, long pepper fruit, longan berries, loofah, loqat leaf, lotus root, lotus seeds, lovage, lycii, ma huang, magnetitum, maitake mushroom, maitake mushroom extract, malva, marjoram, marshmallow, mate, matsutake, mayapple, meadowsweet, melilot, milk thistle, milkweed, mint, momordica chirantia, morinda root, morning glory, morning glory seeds, moutan peony, mucuna pruiens, mugwort, muira puama, mulberry branch, mulberry fruit, mulberry leaf, mulberry root bark, mulberry twig, mullein, mume plum, mushrooms, musk, mustard seeds, myrrh, nasturtium, natto, neem, neem tree, nettle, nettles, nigella, night-blooming cereus, noni, nitric oxide, nopal, notopterygium, nutmeg, oak, olive leaf extract, ophiopogon, orach, orange peel, oregano, oregon grape, oregon grape root, pal shu, panax ginseng, ostrea gigas, parsley root, passion flower, passion fruit, passionflower, pata de vaca, pau d'arco, peach seed, pennyroyal, peony, peppercorn, pepperine, perilla, perilla leaf, phragmites, phytolacca, picorhiza kurroa, pigweed, pinellia, pine tree pollen, pipsissewa, plantain, platycodon, playcodon, pleurisy root, poke, polygala, polyporus, poppy, poria cocos, posole, prickly ash, prickly ivy, prickly pear, privet fruit, prunella, psoralea, pueraria, puncture vine fruit, puncturevine, purslane, pygeum, pyllanthus, pyrola calliantha, quaking aspen, radish seed, rapadura, raspberry leaf, red clover, red peony root, red raspberry, red sage herb, rehmannia, reishi mushroom, rose, rosehips, rue, rutin, sacha jergon, safflower, saffron, saint john's wort, salsify, salt bush, salvia, sandalwood, sanguisorba, sargassum, sarsparilla jamaican, sassafras, saussurea, savory, schisandra, schisandra berries, schizonepeta, scrophularia, scullcap, scutellaria, sea cucumber, sea whip, sedum, senna, senna leaf, shatawari, shi hu, siberian ginseng, sileris, skullcap, slippery elm, solomon's seal, sophora root, sorrel, squawvine, st john's wort, star anise, stephania, stevia, stinging nettle, suma, sundew, sweet annie, sweet basil, sweetflag, talcum, tamarind, tang keui, tarragon, tea, thistles, thuja, thyme, tian qi, tienchi, torreya, trichosanthes fruit, tulsi, to chung, turkey tail, turmeric, twig tea, umeboshi, uncaria stem, usnea, uva ursi, valerian root, vegetable charcoal, vervain, violet, violets, vitex, vitex fruit, walnut fruit, walnut leaves, wheat grass, white mulberry leaf, white oak bark, white peony, wild, carrot, wild cherry bark, wild ginger, wild marjoram, willow, witch hazel, wood betony, wood ear, wormwood, xanthium, yautia, yellow dock, yerba mansa, yerba santa, yerbamate, yohimbe, yohimbine, yucca and Zizyphus, and a method of studying the utilization of a coffee bean and extracts thereof for improving sexual performance comprising obtaining a first ingredient selected from among a coffee bean and an extract thereof; and combining the first ingredient with one or more second ingredients which improve sexual performance and provide one or more animal subjects with measured portions of the first ingredient and the second ingredient, where the one or more animal subjects consume said measured portions by a means selected among eating and drinking and ascertain a degree of improvement in sexual response and adjust an amount of the first ingredients and an amount of the one or more second ingredients in an amount which improves sexual performance and wherein the ingredient selected from among a coffee bean and an extract thereof increases the effectiveness of the one or more second ingredients which improve sexual performance.

### DETAILED DESCRIPTION OF THE INVENTION

It is preferable that herbs and other components of blends are used at their peak freshness. Organic ingredients are also preferable in most cases. However, when herbs and other components of the blend are used for taste, smell, color, synergy and texture, for example, they may be first aged and/or processed for the feature/property desired. Also, they may be used prior to full maturity for a desired property such as taste, smell and color, for example.

Blends of the present invention which provide sexual enhancement are comprised of one or more ingredients and preferably seven to eighteen ingredients. One or more particular blends of the present invention are comprised of seven to twelve ingredients. Particularly notable results were obtained with combinations of twelve ingredients, eleven ingredients, ten ingredients, eight ingredients and seven ingredients. These combinations of ingredients were found to act synergistically to produce the desired results. The seven to twelve ingredients are diluted by the combination of one to another. Additions to these should be done with care so as not to have an undesirable effect on a dilution ratio e.g., exceeding certain synergistic margins. Herbs and other components of the blend of the present invention have medicinal properties that can deteriorate over a period of time and which can also become unstable. In such a case it is preferable to use fresh harvested herbs and other ingredients in certain blends of the present invention. Further, since shelf life of various ingredients of the present inventions is varied, care must be taken when selecting them for use in the blends. Shelf life and spoilage advice for each herb and other components selected should be carefully considered. Some herbs and ingredients of the present invention can be dried. With respect to herbs, drying is the transformation of the harvested leaf and stem, containing about eighty to ninety percent water to a stable state containing about five to ten percent water. It is preferable to minimize a holding time between harvest and drying. Activities of harvest and drying are coordinated in order to prevent delays in drying and to reduce incidents of spoilage. A temperature factor during drying is important and can be critical in some instances. For example, if a drying temperature is too high, then volatile essential oils of some components can be depleted. A valuable portion of some components can be lost altogether. However, a component may be useful for taste, color and smell, for example. In such a case, a property of such components with respect to an active ingredient may be of diminished value.

For many herbs, it is preferable that drying temperatures are below 40° C. with forced air movement. For the present invention, herbs and components used herein include roots, flowers stems and leaves. Some of the herbs are utilized for the content of the leaf part of the plant and others are utilized for the content of the stem or root. Also, valuable components are taken from the flower of some plants. For the present invention, parts or portions of herbs are utilized primarily for taste, color and smell, for example.

### Blend 1

In at least one embodiment of the present invention, ingredients included are choices among Horny Goat Weed, including a component of a leaf thereof, pomegranate seeds, dandelion, Ginseng, including a component of a root thereof, ginger, including a component of a root thereof, carob bean, lady's mantle, cayenne pepper, kola nut, including a component of a fruit thereof, Guarana, black cohosh, L arginine in solid or liquid form, peanut, watermelon and extracts thereof, chaste tree berry, nitric oxide, oatstraw, including a component of a stem and leaf thereof, damiana, including a component of a leaf thereof, horsetail, sarsaparilla including a component of a root thereof, sassafras, saw palmetto, including a component of a fruit thereof, Maca, including a component of a root thereof, suma, parsley, muira puama, flax seeds, Tibullus, including a component of a root thereof, kelp, pedalium murex, DHEA and tonka bean, vanilla grass, and Cnidium momier, including a component of a seed thereof. Other ingredients disclosed herein may be added less for efficacy than for taste, texture, smell and or color. This blend, denoted as blend 1, enhances libido (especially in a male), increases blood flow overall and to reproductive organs (including a penis), increases stamina, including sexual stamina, provides firm and long lasting erections, increases sexual desire. Blend one enhances overall health including health of a male who has had prostate surgery, experienced erectile dysfunction. Blend 1 may be administered in any form available including pill form or in a solid or liquid food.

### Blend 2

In another embodiment of the present invention, hereinafter referred to as Blend 2, the composition includes at least oat straw, ginseng (preferably red ginseng), dandelion, motherwort, chaste tree berry, black cohosh, saw palmetto, parsley, kelp, vanilla bean, raspberry (preferably red raspberry) and sarsaparilla. Blend 2 may be administered in the same forms as Blend 1.

### Blend 3

In another embodiment of the present invention, hereinafter referred to as Blend 3, the composition includes at least lady's mantle, Ginkgo Biloba, Sencha Fuji, crane's bill, cabbage, periwinkle, Rosemary, sage, gota, kola nut, basil (including holy basil) lavender, shepherd's purse, yarrow, black haw, peppermint, chamomile and spearmint. Blend 3 may be administered in at least the same forms as Blend 1.

### Blend 4

In another embodiment of the present invention, hereinafter referred to as Blend 4, the composition includes at least Shepherd's purse, periwinkle, ladys' mantle, yarrow, cranes bill, black haw and peppermint. Blend 4 may be administered in at least the same forms as Blend 1.

### Blend 5

In another embodiment of the present invention, hereinafter referred to as Blend 5, the composition includes at least moringa, green tea component, garlic, hawthorn, chokeberry, ginseng and ginko biloba. Blend 5 may be administered in at least the same forms as Blend 1.

### Methods of Forming Blends

To form the blends and variations thereof, sifted and/or powdered herbs and other constituent ingredients are separated into lots of about equal weights within certain specified variations. This holds true whether a blend contains many components (ingredients) or relatively few components. For a particular effect, including taste, color and smell (for example), a weight of any of these components can be adjusted. It is preferable that a weight of any of the individual components be increased or reduced by an amount no greater than seventy percent (70%). However, if a particular result is desired, then a weight of a component can be reduced or increased by an amount of about ninety percent (90%). For example, if all components in a blend weight 5 lbs (five pounds) each, it is preferable that the 5 lbs adjusted upward weight to no more than 8.5 lbs. On the other hand, it is preferable that the particular component be adjusted downward in weight to no less than 1.5 lbs. In case of the exemplary blend, a maximum effectiveness for impotence and/or erectile dysfunction is obtained when the tolerances are kept within +/−10%.

These specific components in the above preferred embodiment are selected and blended together because of the unique property of each herb and how each they synergistically react to each other in this blend. Other combinations of herbs were attempted, however taste, texture, smell and side effects had to taken into account and these combinations were not preferred. For example, while benefits for one or more of these blends could be obtained from ginkgo and licorice, they were excluded from some blends and included in others because of taste and other undesirable side effects.

In the preferred embodiments of the present invention, the lots are kept separate during an extraction process. In a few cases the lots are kept together. However, keeping the lots separate allows for more accurate accounting of the performance of each component. Also, each component has one or more reactions on its own, besides having one or more subsequent reactions/interactions when combined with other components later or earlier in the process.

Keeping the lots separate during extraction processes reduces incidents of cross contamination and provides means to isolate and observe and correct the amounts and types of each herb or component used. If mixed together the composition of different herbs could compromise the peak warming time and temperature of each herb.

There are several methods of forming a blend and using a blend, including a method that has been distilled to it simplest teaching. All processes disclosed herein can be automated or batch processes and can be subject to variations, all falling within the scope of the present invention.

### First Method of Forming

The process and equipment used herein to instruct how to make and use the invention is exemplary and is by no means meant to be restrictive.

Accordingly, in one embodiment of the invention using an exemplary twelve (12) five pound lots to establish a preferred ratio between selected components, the individual lots are placed into a container having a five (5) gallon capacity (quantities are for purposes or illustration of ratios). For this example, glass containers (including a glass jar) are used. This is for the extraction of desirables from the selected herbs and/or components. Metallic containers can be used in some instances when they have surfaces which do not cause the herbal compound to lose its alkaline or diminish other desirable contributions. In any case, each container is marked and/or labeled. It is preferable that a color coded container with a matching color lid is used either independently of the labeling or in conjunction with labeling. Any reliable form of documentation for identification purposes can be used.

In this simple case where glass jars are used, a thickness of about at least one fourth of one inch is preferred. The thickness provides a temperature insulation property sufficient to provide for the ingredients to heat at a slow and gradual pace where required. This is because surges in temperature or unstable temperatures could interfere with the preservation of valuable medicinal properties which are extremely delicate in some herbs or other components.

After placing the twelve different herbs and components into twelve separate containers, about one gallon of liquid is added to each container (the one gallon is for ratio illustration). It is preferable that the liquid is organic pharmaceutical grade alcohol. However, other suitable alcohols can be used. The strength and descriptions of some of these are disclosed in embodiments described below. For this embodiment, the alcohol may be up to one hundred ninety (190) proof. It is preferable that the liquid is sterile. It is preferable that the liquid possess sterilizing properties. For example glycerin may be used if desired.

Herbs and other components are saturated in the liquid (in this case alcohol). It is preferable that herbs and other components of the blend are completely saturated in the liquid (in this case alcohol). If there are containers or jars in which the herbs are not completely saturated, then add more of a liquid such as water (preferably distilled water) until the herbs and other components are fully saturated. Distilled water is added to further stabilize and insulate during this process. An amount of water added to each jar is dependent upon the absorption rate of the herb in each jar during this process.

Some herb compositions are used as a powder (meaning finely ground) and others are cut, chopped, pulverized and/or sifted. The differing compositions may require that differing amounts of water be added. The temperature of the water being added at this stage is room temperature. In this case, room temperature is about seventy seven (77) degrees Fahrenheit. Ambient pressure for this method is about 14.7 psig.

A next step is to seal the containers. A variety of types of seals can be used. It is preferable that for this exemplary crude method, jars and lids which are threaded and matched by color and/or other forms of labeling.

For this simple method, one or more electrical heating elements at or near the bottom of a tank is used to control heat of the components during this process. It is preferable that in the case of jars, the jars are not allowed to touch each other or to touch the bottom of the tank. Further, care must be taken to prevent direct contact of the jars with a heating element. A cloth or other suitable material which can be selectively seated or positioned in the water can be used to insulate the jars from a heating element. Otherwise, a desired temperature control cannot be maintained.

For this embodiment jars can be suspended in water within a tank by hooks, cables, racks or combinations thereof. Jars can be suspended at various angles. For example, jars can be suspended at an angle of about 45 degrees.

Hooks, ropes, cables or other types of cords can be connected to a grate positioned parallel to the water and jars suspended therefrom. Grates can be constructed of wood (yellow pine, for example), metal, polymer or other suitable material. Cords or cables can be constructed from any suitable material. Teflon cables, for example, can be used. If a metal is used, then it should be a metal which does not inhibit or interfere with extraction or infusion of desired components of the constituents of the herbs or other ingredients.

For this particular method, cables can be attached at the top and center of jars. In one embodiment, the jars are tilted at an angle suitable to provide for submersion of about sixty to one hundred percent of the jar in the water.

An angle of about forty five degrees is suitable to allow for a broader herbal surface to be effected while being brought to a gentle or slow boil. A broad upper surface and narrow bottom causes a funnel effect in the jars. The herbs heat up more evenly and cool more evenly. Even cooling and heating is preferred. A broader surface provides for greater exposure. The herbs percolate/radiate to the top more uniformly.

In a preferred embodiment, a tank or vessel holding the jars is constructed of porcelain or similar material. For example, a material having the same or similar properties of porcelain can be used. For example, a material which maintains and/or dissipates heat at more uniform or more even rates can be used. Once water reaches desired temperatures (at or near boiling) burners or other heating source can be turned off.

For the preferred embodiment, temperatures and pressures are based on atmospheric pressure (14.7 psi). While this method is a very simple method, the present invention includes (does not exclude) methods by which the heating, boiling, cooling, etc. can be accomplished under a vacuum, including a complete vacuum, and also at higher pressures. Also temperatures can be varied according to pressures. Such methods are known in the art and need not be discussed here.

According to the method presently described, allow jars to boil for about two hours. At the temperature and pressure indicated, about two hours allows sufficient separation of desired components from the flower, leaf, stem or root of the herb or other ingredients. The starting temperature should be about 160° F. It is sufficient to allow the temperature to remain at one hundred and sixty (160) degrees Fahrenheit for about thirty (30) minutes. Then, increase temperature to one hundred and eighty degrees (180° F.). Temperature increases are preferably gradual. Allow temperature to remain at about 180° F., for about forty five (45) minutes. Then, increase the temperature to about two hundred degrees (200° F.). Allow the temperature to remain at about 200° F. for about thirty (30) minutes. Then increase the temperature to about two hundred and twelve (212° F.). Under atmospheric pressure, the water will start boiling at a temperature of about 212° F. Allow the water to boil for about two hours. If a vacuum process is used, a boiling point of water at any temperature and pressure is acceptable. In other words, temperature required may be altered according to a linear or other corresponding relationship with pressure.

After this, remove lids from jars and allow jars to rest with lids removed at about one hour or more. It is preferable that the jars are cooled naturally and gradually in the water. This minimizes any potential damage to compounds or other desirable extracts or ingredients.

Rates of cooling depends upon the amount of water, the ambient temperature, relative humidity, elevation, the material of whatever vessel is holding the water, etc. As a rule of thumb, however, jars or other vessels have reached sufficient cooling when they can be handled with bare hands without injury to a person handling them. At this point, contents of the jars or vessels will have settled to a desirable state. Jars can be corporately or individually allowed to cool uncovered in an open environment or in a closed environment.

In a case of a closed environment, an air sample may be obtained and measured for alcohol and other components which are released during the resting and or cooling period. In a case of a closed environment, a measurement of ninety to one hundred percent alcohol in the vapor space is initially acceptable. Measurements taken are at desirable levels when alcohol content in the vapor space is negligible (near 0%). Further, measurements of desirable components in the vapor space are desirable when they comprise about less than 1% by weight or volume of the sample. This control is used to determine stability of the blend. Alternatively, for some uses, an amount of alcohol in the blend may be desirable for an intoxicating or euphoric result. In this case, amounts of alcohol ranging from one to five percent, five to ten percent, or ten to twenty percent may be acceptable in the vapor space.

Depending on the method of measurement, jars may be allowed to cool in the water or out of the water. When jars are cooled while still in the water (lids off), then cables are used lift the jars to a sufficient height for visual inspection of the jars and the contents thereof. A cracked or leaking container will signal a compromise in the integrity and or reliability of the blend. Normally a distance of a portion of the jar and the water of about three to five (3 to 5) inches is acceptable. The jars will cool completely to room temperature while suspended. The jars will cool to room temperature at a different rate than the water in which the herbs were boiled. The air is able to travel around jars evenly while suspended above the water.

After sufficiently cooling, fill each jar (with herbs in the jar) with distilled water. Fill jars just below the brim with the herbs in the jar. Then, reseal jars and lower them back into water, this time it is preferable that jars are hung perpendicular to water tank. Jars are hung perpendicularly in order to reposition the herbs before they are brought back to a boil. Bring jars back to boil under same insulated conditions.

Allow to boil for an additional two hours in order to capture any medicinal trace elements which may have not separated during the first boil. Then remove jars from water tank and place mixtures into a cool environment.

It is preferable that the jars be placed in a dark room or enclosure. It is acceptable to let jars rest and cool at an average temperature of about sixty eight degrees Fahrenheit (68° F.). It is preferable to place jars in dark room to prevent sunlight from breaking down the compounds. Allowing jars to cool gradually and naturally with tops sealed provides for quality absorption of alkaloids from herbs. Otherwise, alkaloids and other valuable medicinal properties of herbs can be lost.

For this particular embodiment, the mixtures in the jars are allowed to age between three and seven days. However, greater than three days is preferable. This range of time for the aging process is up to about 7 days is used because it is approximately how long it takes for all agents in the tincture to mature and bond. Generally, additional ageing time is not needed. However an ageing time of less than seven days may damage the tincture preventing full stabilization of the tincture.

During the aging period, agitate for approximately ten to twenty minutes every hour for sixteen hours of each of the up to seven days. Alternatively, agitate jars about every twelve to eighteen hours for at least about ten to twenty minutes to prevent mold from forming. Incidents and frequency of agitation may be decreased to as much as half the time without deterioration. However, it is preferable to not decrease times by more than thirty five percent.

Upon completion of the aging process, a series of straining the solids from the liquid is commenced. Alternatively, the straining may be accomplished in one step. For example, the solid particulates in the jar may be strained through cheese cloth or other fine cloth or screens and pressed by a pressing means to extract all of the moisture possible. Generally, if there are no particles visible to the naked eye floating or otherwise visible in the tincture, then the straining is sufficient.

The next step is to combine the contents of each jar into one or more vessels. In a case of one vessel, about thirty minutes to one hour of vigorous mixing is adequate. On the other hand, automated mixing or mixing by powerful machinery can reduce the mixing time significantly. Mixing is sufficient when the appearance of the blend is homogeneous. There should be uniform patterns visible throughout the mixture.

Medium scale production for the exemplary embodiment having the exemplary components described above, the blend is combined (may be poured) with about nine hundred to one thousand (900 to 1000) pounds of coffee beans or particles thereof. About nine hundred fifty to nine hundred seventy (950 to 970) pounds is preferable. It is preferable that the coffee beans are organically grown. It is preferable that the coffee beans are roasted. It is even more preferable that the beans are freshly roasted. Freshly roasted beans are preferred for infusion process because they are still porous and flavors are at their peak. The freshly roasted beans work best when they are cooled. It is preferable that they are naturally cooled. Coffee beans normally will cool overnight after roasting. The average temperature twenty four hours after a roast is approximately ninety two degrees Fahrenheit (92° F.). This is preferred for infusion according to the present embodiment. However, should there be a longer delay after the beans are roasted, for example one or two weeks, this should be acceptable. Any longer than two (2) weeks after roasting can make the infusion process less effective.

For this exemplary embodiment, a mix ratio of about six to ten (6 to 10) ounces of liquid mixture to every about one pound of coffee beans is effective. However, four to six (4 to 6) ounces is preferable. At this ratio, practically all of the beans are thoroughly saturated and possible mold is inhibited. It is preferable that these amounts are varied no more than by twenty percent (20%). A twenty percent variation has degrees of effectiveness. A ten percent variation has degrees of effectiveness. However, the amounts can be varied and an effective process can be maintained within these boundaries.

It is also acceptable for the beans and the blend be combined by any method that will ensure maximum absorption of the blend by the beans. For this embodiment, the mixture and the beans are allowed to rest together in a container for a period of time sufficient for maximum absorption of the blend into the beans. In a case of an open vessel, any residual alcohol or combustibles can be released during infusion. The infusion process can also be accomplished with a closed vessel under pressure. If there is need for volatiles such as alcohol to be released, then the vessels can be opened to atmosphere or to a lesser pressure environment later (after infusion, for example). For this preferred embodiment, the infusion process takes between 6 and 24 hours depending on amount of liquid, size of beans, content of liquid, temperature and pressure. Under ambient conditions, eight to twelve hours is effective. At some rates, overnight is preferable.

After the time for infusion expires, the beans are subjected to a drying process. For drying, it is preferable that the beans be spread out in a thin layer. In a simple form, the beans are spread out over a material such as glass (can be glass or glass-like screen e.g. perforated glass). Beans can be spread out over a suitable mesh or screen to allow air flow from multiple directions. Materials other than glass can be used as long as it is suitable for preventing beans from coming into contact with metallic or other less desirable surfaces during infusion process. Metallic surfaces are rarely recommended because they can leach out alkaloids and other desirable components from beans after infusion.

In a case where the beans are spread out on sheets of acceptable materials, the sheets can be placed on tables or other surfaces which provide vibration or oscillation or some other form of movement of the beans. The beans will release liquids and gases during this process. Further, beans can be placed under fans or subjected to a moderate air flow across the surfaces. Using this method, drying can be achieved about between twenty four and seventy two hours. It is preferable that the drying process be allowed about forty eight hours. Drying can also be accomplished in a closed environment with sufficient head space to release liquids and gases.

When the beans are sufficiently dry, they are placed in containers. It is preferable that the beans be placed in one or more non-metallic containers.

In reiteration of the above described exemplary method, the method employs a recipe using about forty to eighty gallons (preferably sixty gallons) of herbal extract and about between nine hundred and one thousand pounds beans. Preferable amount of beans is nine hundred and sixty. This ratio is maintained when scaling up or scaling down. It is preferable that the beans are roasted. It is preferable that the beans are organic. The blend is effective when using grade one (grade1) medium roast coffee beans.

The infused beans are preferably ground, placed in a conventional apparatus for preparing coffees, teas or other drinks, and served. The beans can also be packaged in the whole beans state or in various particle sizes of ground coffee beans. The essence of the coffee beans can be extracted and then subjected to a drying process. Drying processes in order of preference are lyophilized, spray dried and then air dried. Particle sizes of dried product can range between 1 and 10,000 microns. It is preferable for most end uses that the powder granular size is between 4 and 600 microns. The drying processes are known to those skilled in the art. Alternatively, the beans and the liquid blend can be dried separately. The liquid blend can be mixed with the pre-dried liquid bean tincture and the two can be dried together. The liquid herbal blend can be combined with a liquid or solid and administered to an animal (including a human animal).

### Second Method of Forming

A second method of forming a composition according to the present invention is to select and obtain herbs and other ingredients as described above. Inspect the herbs and ingredients for undesirable particles. With a grinding apparatus, grind the components to a small mesh particle size. A particle size of about fifty (50) mesh is acceptable. Variations in mesh sizes may be plus or minus twenty percent (20%). Label the components and store them in a dark dry place away from direct sunlight.

Obtain and apply heat to a water bath container having an inner and outer jacket. Any variation of a water bath container may be used as long as it has a water level in a first vessel sufficient to surround a second vessel inside of the first vessel. Heat the container (outer water) to a desired temperature and maintain that temperature. The inner water jacket should be between forty and sixty degrees centigrade. Fifty degrees centigrade is preferable. Document the temperature of the inner water jacket. It should be within the specified range. Place a clean container in the inner water pool. A serial extract glass container may be used.

The following quantities are exemplary (for ratio sake) and may be scaled up or down, with the same ranges and tolerances as the exemplary nominal fifty pound quantifies used in the examples above. Weigh about ten grams of dry quantities of each of the components and place them in a mixer. The mixer should be capable of yielding a homogenous dry mix. It should be cleaned and sterilized before using it. Using eleven (11) components for example, a dry mix should weight about one hundred and ten (110) grams, for example. About five to ten minutes of mixing is usually adequate. Six minutes is adequate for many blends. For this exemplary embodiment, add about five hundred (500) milliliters of water (preferable distilled water) to the nominal 110 grams of dry mix. Add about thirty (30) milliliters of alcohol to the dry mix. Alcohol may be ethanol or isopropyl alcohol (for example) and should have a strength of about fifty to one hundred percent (50% to 100%). Adequate results can be obtained with alcohol seventy percent (70%). Mix the water and alcohol with the dry herbs thoroughly. Four to eight minutes of mixing is sufficient. Acceptable results can be obtained with about five minutes of mixing.

Prepare a gauze net with a mesh size capable or retaining the mixture of water and alcohol and dry ingredients (now wet ingredients) with minimal liquid drip. Put the mixture into the gauze net and secure the net. Cover the inner and outer portions of the water bath container and maintain temperature below fifty degrees centigrade. Agitate the mixture every four to eight hours (preferably about every six hours). Keep covered and away from direct sunlight for about twenty four to sixty hours (thirty six to forty eight hours is acceptable). Document and label the mixture including date of production and note an expiration date or used best by date of about six months.

Perform the next steps in an environment where the room air circulation is four to eight times per hour. Five to seven times per hour is acceptable. Maintain a room temperature of about seventy to seventy five degrees Fahrenheit and a humidity of about forty five (45) rh (relative humidity).

For the filtration process, remove the net with the mixture out of the incubation container (bath container). Secure the open end of the gauze to form a sack configuration. Place the sack into a clean container and allow it to cool down to ambient. A glass container is suitable. Using a mechanism to apply pressure to the sack, squeeze the liquid contents of the gauze sack into the container. This may be done by hand or by an apparatus designed to squeeze. The gauze sack should be squeezed until the pressure applied yields no more liquid (or very little liquid). This process is to make sure that everything that can be captured from the mixture is captured and retained. In a variation or even preferred method of performing the squeezing step, the dry mixture may be placed in a centrifuge to perform the extract.

A remainder (the majority portion) of the valuable extract is still in the incubation container. The next step is to combine the squeezed out liquid to the liquid remaining in the incubation container in the glass container (glass is for example).

After combining the liquid extracts, they will now be subject to filtration through one or more of a series of filters beginning with coarse filters and progressing to fine mesh filters. It is also acceptable to pass the liquid through only one or more fine mesh filters.

Preferably, the liquid extract is passed through a ⅜″ diameter on 9/16″ filter center, then ¼″ diameter on ⅜″ center, the ⅛″ diameter on 3/16″ center and then 1/16″ diameter on ⅛ center. This step in the filtration process can be referred to collectively as filter one, and either one or the entire combination of filters may be successively employed.

Filter two comprises passing the liquid extract through a 4×4 mesh and then an 8×8 mesh. One or the other may be used. However, in some cases, this step can be eliminated. Filter three comprises passing the liquid extract through a 20×20 mesh, a 40×40 mesh and then through a 60×60 mesh. Multiple successive filtering steps are preferable, but may be reduced or modified.

The liquid extract is now suitable for use as a liquid and for human consumption as a liquid. The liquid extract can be combined with another liquid to form a drink such as a sports drink or a beverage (coffee included). The liquid extract can be combined with a solid or semi-solid food such as a cookie, candy or chewing gum, for example. The liquid extract may be eaten or drank and can be consumed by any suitable, safe and desirable method.

The liquid extract may also be dried (by know methods) in a further step in order to obtain a dry powder. The dry powder is preferably and usually water soluble and can be used in a variety of applications for administration. These applications for administration include tablets or pills, instant coffee blends, other water soluble blends, sports drink additives, candy, chewing gum or any other form of food or drink. The use of the liquid or dried powder is not meant to be limited by the examples described herein. Any method of delivery which eases the difficulty of administration and compliance is contemplated herein.

### Third Method of Forming

The third method of forming is basically the same as the second method of forming with few exceptions. Particularly, rather than combine all of the dry herbs together, each dry herb is subjected to the extraction process separately. Ratios of liquids to dry ingredients should be maintained and adjusted according to weight.

### Role of Chromatographic Analyses

The various blends of the present invention took into account that the multiplicity of components in the blends are subject to heating and cooling. Consequently, reactions occur which alter the original compositions. There are reactions that nullify the efficacy of some components of the blends. There are reactions that enhance the efficacy of some components of the blends. It also appears that that entirely different compounds result from the processes which are employed in making the blends. Therefore as a component (including an herbal component) selection measure and a quality control measure, the inventors employ a method of running many spectrums (also referred to as chromatograms) of various blends in order to minimize duplication of apparent same or similar components, and also to eliminate components which react to diminish desirable components. The analyses are performed on instrumentation (chromatographs) which have column/valve arrangements which provide for separation of components without the necessity of identifying what the actual components are. Chromatograms are run separately for each liquid extract for each component. Chromatograms are then compared to see which components elute at relatively the same time. Based on the proximity of the elution of certain components in each of the ingredients, it can be determined that they likely exhibit the same or similar properties and produce similar benefits. Chromatograms are overlaid and also time stamped for comparison. Preferably peak height analysis is used, but peaks subject to integration beneath the curve (peak area) is also employed in certain situation. With this method, herbs having spectrums which indicate high concentrations of beneficial compounds may be added together for increased effect, or eliminated or reduced in order to avoid unnecessary duplication. These chromatograms (spectrums) are retained for quality control. Compounds or components which are not subject to identification are given fictitious names and are used for quality control for the entire process. For example, if component number 3, 4 and 5 from the ginkgo extract yields quantities of 13, 22 and 63 parts per million under the process for a current production run, it should yield relatively the same results for successive production runs. Otherwise, it indicates that something is lacking.

While the chromatographic methods employed are time consuming, they provide the quality controls needed to insure continued effectiveness of the products of the present invention. In most applications, boiling point columns are used, thus making the applications broad and simple. For most applications, gas chromatograph with TC (thermal conductivity) detector is used. Carrier gas is helium or nitrogen in most cases. Flow rates and temperatures are initially set high in order for a totality of components in an herb, ingredient or blend to elute quickly. A tentative result is obtained by quantifying the resultant peak or peaks as 100%. Lower temperatures are then employed in order to achieve separation of components. The chromatogram is then labeled with an identifier such as “Chromatogram for Ginseng Root”, for example. Other selected components are then run on the same column and with the same parameters. Components eluting at the same time are assumed to have properties similar to those of the exemplary ginseng root. Several components are run together in order to see whether peaks are diminished, increased or eliminated altogether. Selections of ingredients are based on the results obtained.

The process for testing the efficacy of the blends has been time consuming and has been in development for more than three years.

Participants in the study and development of the blends have ranged from male subjects from age thirty to age seventy five. Some participants who had not been sexually active for numbers of years experienced restoration of libido. Some participants had no prostate at all. A number of participants had nerve sparing surgery and a number of them did not. Some comments made by participants were “I am like a seventeen year old again” and “my wife can't take it any more”. Spouses and sexual partners also have affirmed that the blends are effective. Sustainable hard erections of at least thirty minutes are commonly reported. Some have reported erections lasting up to one hour during intercourse.

### Benefits of Blends

All of the blends of the present invention were reported to have the benefits of positive mood enhancement, positive outlook, clarity of thought processes, increase in creative thinking, reduction in fatigue, energy without jittery feeling (even with caffeine), improved sleep, increase in blood circulation, increase in libido, increase in sexual desire, improvement in sexual performance, control of appetite (including reduction in episodes of overeating), calmness, reduction in anxiety, reduction in depression, elimination of fearful outlook.

Blend number one has added benefits of improving sexual performance, longer and harder penile erections in men, spontaneous erections, increased sexual desire, sustained penile erections during sexual intercourse including erections of durations of twenty minutes, twenty five minutes, thirty minutes, forty minutes, and fifty minutes during a single event of sexual intercourse. Additional benefits include sustained penile erections of durations of twenty minutes, twenty five minutes, thirty minutes, forty minutes, and fifty minutes during a second and even third event of sexual intercourse during a period of five consecutive hours. These benefits are obtained in the absence prescription drugs with the same or similar actions as Viagra, Cialis and Levitra. Blend one enhances blood flow and stamina. Blend one delivers critical nutrients to aid in the prevention and recover of prostate cancer and erectile dysfunction.

In addition to the above described benefits, blend number two has added benefits of improving sexual performance, soothing menstrual cramps, hot flashes and improving and enhancing libido.

Blend number three also enhances digestive efficiency. It delivers critical nutrients that increase the metabolic rate at which the body metabolizes food.

Blend number four also enhances cognitive functioning. It delivers critical nutrients that increase positivity, focus and mental agility.

The delivery method of the blends is selected to coincide with normal and usual daily activities. Such activities include drinking water and drinking coffee or tea. Such methods of delivery or administration provide an easy to realize incentive for taking the nutrient. It overcomes the cumbersome and often discouraging task of remembering to take your vitamins or medicines. The invention includes combining the blends with favorite foods and beverages to increase compliance with recommended dosages.

### Dosage

Effective dosages for powder form (including a lyophilized powder) for all blends are between about 150 and 250 milligrams per day. Preferable dosage is about 200 milligrams per day. More preferable is about 100 milligrams twice per day. For liquid extract form, the dosage is about three to six milliliters twice per day. This can be added to a desired food or drink. In a case of coffee, for example, the indicated amount of liquid extract can be added to a cup of coffee between four and eight ounces. The coffee may be either caffeinated or decaffeinated. Caffeinated coffee does not interfere with effectiveness of blends and does not cause jitteriness or interfere with normal sleep. A temperature of consumption and preparation is determined to impact effectiveness of the blends. There is increasing benefit at temperatures which the blends are consumed. Benefits are provided by cold consumption. A low but adequate benefit was provided by consumption at near freezing (that is near 32 degrees Fahrenheit). Increasing benefit was obtained at a temperature range (all Fahrenheit) between 40 and 80 degrees, 80 and 100 degrees, 100 and 140 degrees, and 140 and 200 degrees. Positive enhancement effects and affects were reported to be obtained more rapidly at higher temperature ranges. Results claimed herein were confirmed by observation and reports of participants who consumed the blends over the years of experimentation.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated. It is understood that various omissions, substitutions of equivalents are contemplated as circumstance may suggest or render expedient, but is intended to cover the application or implementation without departing from the spirit or scope of the claims of the present invention.

What is claimed is:

1. A synergistic blend for benefiting health and improving sexual performance in an animal comprising:
one or more components selected from among horny goat weed and pomegranate seeds and dandelion;
one or more components selected from among a group comprising various forms of ginseng, the forms including asian ginseng, siberian ginseng, red white Ginseng and american ginseng;
one or more components selected from among ginger root, carob bean, lady's mantle and cayenne pepper;
one or more components selected from among peanuts, extract of watermelon rind, l-arginine, chaste tree berry and nitric oxide;
one or more components selected from among black cohosh, guarana and kola nut;
one or more components selected from among oatstraw and horsetail;
one or more components selected from among damiana leaf and red raspberry;
one or more components selected from among sarsaparilla root and sassafras root;
one or more components selected from among flax seeds and saw palmetto;
one or more components selected from among maca root, suma, parsley and muira puama;
one or more components selected from among tribulus terrestres kelp pedalium murex and DHEA; and
one ore more components selected from among cnidium momier, tonka bean and vanilla grass.

2. The blend of claim 1, where the blend comprises one or more of herbal and non herbal extracts and components, wherein the blend is infused into one or more of a solid and semi-solid medium, said medium including one or more of a coffee bean and a part thereof, said coffee bean and part thereof being one or more of caffeinated and non-caffeinated, and
wherein said blend is administered as one or more of a liquid, a solid and a semi solid, and
wherein an administration of said blend to a consumer provides one or more health benefits to a consumer, the health benefits including maintaining a penile erection for a duration of time.

3. A synergistic blend for benefiting health and improving sexual performance in an animal comprising:
one or more components selected from among gota and sage;
one or more components selected from lady's mantle and gingko biloba;
one or more components selected from among crane's bill and sencha fuji;
one or more components selected from among cabbage and kola nut;
one or more components selected from among periwinkle and rosemary;
one or more components selected from among yarrow and holy basil;
one or more components selected from among shepherd's purse and lavender;
one or more components selected from among black haw and chamomile; and
one or more components selected from among peppermint and spearmint.

4. The blend of claim 3, where the blend comprises one or more of herbal and non herbal extracts and components, wherein the blend is infused into one or more of a solid and semi-solid medium, said medium including one or more of a coffee bean and a part thereof, said coffee bean and part thereof being one or more of caffeinated and non-caffeinated, and
wherein said blend is administered as one or more of a liquid, a solid and a semi solid, and
wherein an administration of said blend to a consumer provides one or more health benefits to a consumer thereof, the health benefits including maintaining a penile erection for a duration of time.

5. The blend of claim 1, wherein the blend is administered in combination with a favorite food or beverage.

6. The blend of claim 3, wherein the blend is administered in combination with a favorite food or beverage.

7. The composition of claim 1, wherein additional ingredients having a benefit selected from among taste, smell, color, synergy and texture are selected from among one or more of abalone shell, abizzia bark, abuta, acanthopanax. aconite, agastache, agrimony, akebia, alfalfa, alcohol, alisma, aloe vera, amalaki, amasake, american ginseng, amla, andrographis paniculata, anemarrhena, angelica, angelica du huo, anise, aniseed, annato, apricot seed, aquilaria, aralia, arisaema flower, arnica, artemisia annua, artemsisia annua, artist conk, asafetida, asafoetida, ashwagandha, asparagus fern root, asparagus root, asparagus shoots, astragalus, astralagus, atractylodes, pueraria lobata root, gaba, wild yam extract, muira puama extract, HCl, vitamin b6, eleutherococcus extract, xanthroparmelia scarbrosa, velvet deer antler, pumpkin seed, stinging nettle, astragalus, licorice extract, ho shou wu extract, hops, zinc, and boron; saffron, tumeia aphrodisiacs, irish moss, and fake unicorn root. if desired, the composition may additionally include one or more other vitamins or minerals, ginseng, panax ginseng, red raspberry, curculigo, mucuna pruiens, ostrea gigas, pyrola calliantha, cuscuta chinensis, dong quai, cynomorium songaricum, bitter melon, momordica, american ginseng, bilberry, vitamin e, DHEA, balm of gilead, baptisia, barberry, bay leaf, bayberry, begonia, beleric myrobalan, benzoin, bilberry, biota leaves, biota seeds, bitter melon, bitter melon juice, bitter orange, black atractylodes, black cumin, black currant seed oil, black current, black nightshade, black sesame seed, black sesame seeds, black soybean, blackberry lily, bladderwrack, blessed thistle, bletilla, bloodroot, blue cohosh, blue flag, blue vervain, bogbean, boldo, bolete, boneset, borage, borneol, boswellia, bovine tracheal cartilage, brahmi, buchu, buckthorn, buddleai flower, bugleweed, buglweed, bupleurum, bupleurum root, burdock, burdock root, butcher's broom, butcher's-broom, cajueiro fruit, cajuput, calamus, calendula, california poppy, camellia, camomile, caraway, cardamom, cascara sagrada, cassava, cassia seed, castor oil plant, castor-oil plant, catha, catnip, cat's claw, cattail pollen, cayenne pepper, ceanothus, celandine, celeriac, cernilton, cernitin, chanca piedo, chaparral, chebulic myrobalan, chelidonium, chelone, chenopodium, cherry stems, chickweed, chicory, chinaberry bark, chinese angelica, chinese quince, chinese raspberries, chinese thoroughwax, chinese water melon, chrysanthemum, chuchuasi, cibotium, cijuwa, cimicifuga, cinnabar, cinnamon, cinnamon twig, cistanche, citrus peel, cleavers, clematis root, cocklebur fruit, codonopsis, coix, collinsonia, coltsfoot, coltsfoot flower, comfrey, coptis, cordyceps, corn silk, cornsilk, corydalis, cow bezoar, cramp bark, cranesbill root, cress, culverts root, cumin, curculigo, curcumin, cuscutu cheninsis, cuttlefish bone, cyperus, cynomorium songaricon, cypress, dahlia, day lily, deer antler, deglycyrrhizinated licorice, dendrobium orchid, devil's claw, dhea, devils' club, dill, dill weed, dioscorea, dipsacus root, dogwood berries, don sen, dong quai, drynaria, echinacea, eclipta, elder, elder bark, elderberry, elderberry extract, elderflower, elecampane, eleuthero, emblic myrobalan, enoki, epazote, ephedra, epimedium, epimedium leaf, eucalyptus, eucommia bark, euphorbia, ansui, euryale seeds, evening primrose, eyebright, false unicorn, fennel, fennel seed, fenugreek, feverfew, fleeceflower, fleeceflower, stem, florence fennel, forsythia, forsythia fruit, fritillaria, fo-ti, fu ling, fruit juice, galangal, garcinia, gardenia fruit, garlic, garlic chives, gastrodia, gentian, gentian root, geranium, ginkgo nut, ginseng, glehnia root, golden seal, goldenrod, goldenseal, goti berries, gota kola, gotu kola, grapefruit seed extract, gravel root, green tangerine peel, green tea, green tea extract, grindelia, ground ivy, guaiacum, guarana, guggul, gugl, gugulipid, gymnema sylvestre, gypsum, hato mugi, hawthorn berries, hawthorn berry, hemp seed, hercampure, hermatitum, hibiscus, ho shou wu, honeysuckle, honeysuckle flowers, hops, horehound, horse chestnut, horse, hestnuts, horseradish, horsetail, houttuynia, huckleberries, huitlacoche, iceland moss, immature bitter orange, impatiens, imperata, inula flower, ipecacuanha, isatis root, japanese elderberry, japanese knotweed, japanese turf lily, jasmine, jatoba tree, jinengo, jujube date, juniper berries, kava kava, khella, knotweed, kudzu, kuzu root, lady's slipper, lantana, large-leafed gentiana, laver, ledebouriella, lemon balm, lemon grass, licorice root, ligusticum, ligustrum berries, ligustrun, lily bulb, linden flowers, lindera root, litchi, lo han kuo, lobelia, lomatium, long pepper fruit, longan berries, loofah, loqat leaf, lotus root, lotus seeds, lovage, lycii, ma huang, magnetitum, maitake mushroom, maitake mushroom extract, malva, marjoram, marshmallow, mate, matsutake, mayapple, meadowsweet, melilot, milk thistle, milkweed, mint, momordica chirantia, morinda root, morning glory, morning glory seeds, moutan peony, mucuna pruiens, mugwort, muira puama, mulberry branch, mulberry fruit, mulberry leaf, mulberry root bark, mulberry twig, mullein, mume plum, mushrooms, musk, mustard seeds, myrrh, nasturtium, natto, neem, neem tree, nettle, nettles, nigella, night-blooming cereus, noni, nitric oxide, nopal, notopterygium, nutmeg, oak, olive leaf extract, ophiopogon, orach, orange peel, oregano, oregon grape, oregon grape root, pai shu, panax ginseng, ostrea gigas, parsley root, passion flower, passion fruit, passionflower, pata de vaca, pau d'arco, peach seed, pennyroyal, peony, peppercorn, pepperine, perilla, perilla leaf, phragmites, phytolacca, picorhiza kurroa, pigweed, pinellia, pine tree pollen, pipsissewa, plantain, platycodon, playcodon, pleurisy root, poke, polygala, polyporus, poppy, poria cocos, posole, prickly ash, prickly ivy, prickly pear, privet fruit, prunella, psoralea, pueraria, puncture vine fruit, puncturevine, purslane, pygeum, pyllanthus, pyrola calliantha, quaking aspen, radish seed, rapadura, raspberry leaf, red clover, red peony root, red raspberry, red sage herb, rehmannia, reishi mushroom, rose, rosehips, rue, rutin, sacha jergon, safflower, saffron, saint john's wort, salsify, salt bush, salvia, sandalwood, sanguisorba, sargassum, sarsparilla jamaican, sassafras, saussurea, savory, schisandra, schisandra berries, schizonepeta, scrophularia, scullcap, scutellaria, sea cucumber, sea whip, sedum, senna, senna leaf, shatawari, shi hu, siberian ginseng, sileris, skullcap, slippery elm, solomon's seal, sophora root, sorrel, squawvine, st john's wort, star anise, stephania, stevia, stinging nettle, suma, sundew, sweet annie, sweet basil, sweetflag, talcum, tamarind, tang keui, tarragon, thistles, thuja, thyme, tian qi, tienchi, torreya, trichosanthes fruit, tulsi, tu chung, turkey tail, turmeric, twig tea, umeboshi, uncaria stem, usnea, uva ursi, valerian root, vegetable charcoal, vegetable juice, vervain, violet, violets, vitex, vitex fruit, walnut fruit, walnut leaves, water, wheat grass, white mulberry leaf, white oak bark, white peony, wild, carrot, wild cherry bark, wild ginger, wild marjoram, willow, witch hazel, wood betony, wood ear, wormwood, xanthium, yautia, yellow dock, yerba mansa, yerba santa, yerbamate, yohimbe, yucca and zizyphus.

8. A method of studying the utilization of a coffee bean and extracts thereof for improving sexual performance comprising:
obtaining a first ingredient selected from among a coffee bean and an extract thereof; and
combining the first ingredient with one or more second ingredients which improve sexual performance;
provide one or more animal subjects with measured portions of the first ingredient and the second ingredient, where the one or more animal subjects consume said measured portions by a means selected among eating and drinking;
ascertain a degree of improvement in sexual response; and
adjust an amount of the first ingredients and an amount of the one or more second ingredients in an amount which improves sexual performance.

9. The method of claim 8, wherein the ingredient selected from among a coffee bean and an extract thereof increases the effectiveness of the one or more second ingredients which improve sexual performance.

10. The method of claim 8, wherein an effectiveness of the portions for improving sexual performance is enhanced by said first ingredient without negative effects of constituents contained therein including jitteriness, inability to sleep, crashing and speeding from the constituents including caffeine.

11. The composition of claim 1, wherein an effectiveness of the blends for improving sexual performance is enhanced by an ingredient containing caffeine without negative effects of constituents contained therein including jitteriness, inability to sleep, crashing and speeding.

12. The composition of claim 3, wherein an effectiveness of the blends for improving sexual performance is enhanced by an ingredient containing caffeine without negative effects of constituents contained therein including jitteriness, inability to sleep, crashing and speeding.

13. The composition of claim 3, wherein additional ingredients having a benefit selected from among taste, smell, color, synergy and texture are selected from among one or more of abalone shell, abizzia bark, abuta, acanthopanax. aconite, agastache, agrimony, akebia, alfalfa, alcohol, alisma, aloe vera, amalaki, amasake, american ginseng, amla, andrographis paniculata, anemarrhena, angelica, angelica du huo, anise, aniseed, annato, apricot seed, aquilaria, aralia, arisaema flower, arnica, artemisia annua, artemsisia annua, artist conk, asafetida, asafoetida, ashwagandha, asparagus fern root, asparagus root, asparagus shoots, astragalus, astralagus, atractylodes, pueraria lobata root, gaba, wild yam extract, muira puama extract, HCl, vitamin b6, eleutherococcus extract, xanthroparmelia scarbrosa, velvet deer antler, pumpkin seed, stinging nettle, astragalus, licorice extract, ho shou wu extract, hops, zinc, and boron; saffron, tumeia aphrodisiaca, irish moss, and fake unicorn root. if desired, the composition may additionally include one or more other vitamins or minerals, ginseng, panax ginseng, red raspberry, curculigo, mucuna pruiens, ostrea gigas, pyrola calliantha, cuscuta chinensis, dong quai, cynomorium songaricum, bitter melon, momordica, american ginseng, bilberry, vitamin e, DHEA, balm of gilead, baptisia, barberry, bay leaf, bayberry, begonia, beleric myrobalan, benzoin, bilberry, biota leaves, biota seeds, bitter melon, bitter melon juice, bitter orange, black atractylodes, black cumin, black currant seed oil, black current, black nightshade, black sesame seed, black sesame seeds, black soybean, blackberry lily, bladderwrack, blessed thistle, bletilla, bloodroot, blue cohosh, blue flag, blue vervain, bogbean, boldo, bolete, boneset, borage, borneol, boswellia, bovine tracheal cartilage, brahmi, buchu, buckthorn, buddleai flower, bugleweed, buglweed, bupleurum, bupleurum root, burdock, burdock root, butcher's broom, butcher's-broom, cajueiro fruit, cajuput, calamus, calendula, california poppy, camellia, camomile, caraway, cardamom, cascara sagrada, cassava, cassia seed, castor oil plant, castor-oil plant, catha, catnip, cat's claw, cattail pollen, cayenne pepper, ceanothus, celandine, celeriac, cernilton, cernitin, chanca piedo, chaparral, chebulic myrobalan, chelidonium, chelone, chenopodium, cherry stems, chickweed, chicory, chinaberry bark, chinese angelica, chinese quince, chinese raspberries, chinese thoroughwax, chinese water melon, chrysanthemum, chuchuasi, cibotium, cijuwa, cimicifuga, cinnabar, cinnamon, cinnamon twig, cistanche, citrus peel, cleavers, clematis root, cocklebur fruit, codonopsis, coix, collinsonia, coltsfoot, coltsfoot flower, comfrey, coptis, cordyceps, corn silk, cornsilk, corydalis, cow bezoar, cramp bark, cranesbill root, cress, culver's root, cumin, curculigo, curcumin, cuscutu cheninsis, cuttlefish bone, cyperus, cynomorium songaricon, cypress, dahlia, day lily, deer antler, deglycyrrhizinated licorice, dendrobium orchid, devil's claw, dhea, devils' club, dill, dill weed, dioscorea, dipsacus root, dogwood berries, don sen, dong quai, drynaria, echinacea, eclipta, elder, elder bark, elderberry, elderberry extract, elderflower, elecampane, eleuthero, emblic myrobalan, enoki, epazote, ephedra, epimedium, epimedium leaf, eucalyptus, eucommia bark, euphorbia, ansui, euryale seeds, evening primrose, eyebright, false unicorn, fennel, fennel seed, fenugreek, feverfew, fleeceflower, fleeceflower, stem, florence fennel, forsythia, forsythia fruit, fritillaria, fo-ti, fu ling, fruit juice, galangal, garcinia, gardenia fruit, garlic, garlic chives, gastrodia, gentian, gentian root, geranium, ginkgo nut, ginseng, glehnia root, golden seal, goldenrod, goldenseal, goti berries, gota kola, gotu kola, grapefruit seed extract, gravel root, green tangerine peel, green tea, green tea extract, grindelia, ground ivy, guaiacum, guarana, guggul, gugl, gugulipid, gymnema sylvestre, gypsum, hato mugi, hawthorn berries, hawthorn berry, hemp seed, hercampure, hermatitum, hibiscus, ho shou wu, honeysuckle, honeysuckle flowers, hops, horehound, horse chestnut, horse, hestnuts, horseradish, horsetail, houttuynia, huckleberries, huitlacoche, iceland moss, immature bitter orange, impatiens, imperata, inula flower, ipecacuanha, isatis root, japanese elderberry, japanese knotweed, japanese turf lily, jasmine, jatoba tree, jinengo, jujube date, juniper berries, kava kava, khella, knotweed, kudzu, kuzu root, lady's slipper, lantana, large-leafed gentiana, laver, ledebouriella, lemon balm, lemon grass, licorice root, ligusticum, ligustrum berries, ligustrun, lily bulb, linden flowers, lindera root, litchi, lo han kuo, lobelia, lomatium, long pepper fruit, longan berries, loofah, loqat leaf, lotus root, lotus seeds, lovage, lycii, ma huang, magnetitum, maitake mushroom, maitake mushroom extract, malva, marjoram, marshmallow, mate, matsutake, mayapple, meadowsweet, melilot, milk thistle, milkweed, mint, momordica chirantia, morinda root, morning glory, morning glory seeds, moutan peony, mucuna pruiens, mugwort, muira puama, mulberry branch, mulberry fruit, mulberry leaf, mulberry root bark, mulberry twig, mullein, mume plum, mushrooms, musk, mustard seeds, myrrh, nasturtium, natto, neem, neem tree, nettle, nettles, nigella, night-blooming cereus, noni, nitric oxide, nopal, notopterygium, nutmeg, oak, olive leaf extract, ophiopogon, orach, orange peel, oregano, oregon grape, oregon grape root, pai shu, panax ginseng, ostrea gigas, parsley root, passion flower, passion fruit, passionflower, pata de vaca, pau d'arco, peach seed, pennyroyal, peony, peppercorn, pepperine, perilla, perilla leaf, phragmites, phytolacca, picorhiza kurroa, pigweed, pinellia, pine tree pollen, pipsissewa, plantain, platycodon, playcodon, pleurisy root, poke, polygala, polyporus, poppy, poria cocos, posole, prickly ash, prickly ivy, prickly pear, privet fruit, prunella, psoralea, pueraria, puncture vine fruit, puncturevine, purslane, pygeum, pyllanthus, pyrola calliantha, quaking aspen, radish seed, rapadura, raspberry leaf, red clover, red peony root, red raspberry, red sage herb, rehmannia, reishi mushroom, rose, rosehips, rue, rutin, sacha jergon, safflower, saffron, saint john's wort, salsify, salt bush, salvia, sandalwood, sanguisorba, sargassum, sarsparilla jamaican, sassafras, saussurea, savory, schisandra, schisandra berries, schizonepeta, scrophularia, scullcap, scutellaria, sea cucumber, sea whip, sedum, senna, senna leaf, shatawari, shi hu, siberian ginseng, sileris, skullcap, slippery elm, solomon's seal, sophora root, sorrel, squawvine, st john's wort, star anise, stephania, stevia, stinging nettle, suma, sundew, sweet annie, sweet basil, sweetflag, talcum, tamarind, tang keui, tarragon, thistles, thuja, thyme, tian qi, tienchi, torreya, trichosanthes fruit, tulsi, tu chung, turkey tail, turmeric, twig tea, umeboshi, uncaria stem, usnea, uva ursi, valerian root, vegetable charcoal, vegetable juice, vervain, violet, violets, vitex, vitex fruit, walnut fruit, walnut leaves, water, wheat grass, white mulberry leaf, white oak bark, white peony, wild, carrot, wild cherry bark, wild ginger, wild marjoram, willow, witch hazel, wood betony, wood ear, wormwood, xanthium, yautia, yellow dock, yerba mansa, yerba santa, yerbamate, yohimbe, yucca and zizyphus.

13. The method of claim 8, wherein the one or more second ingredients include ingredients selected from among moringa, green tea component, garlic, hawthorn, chokeberry, ginseng and ginko biloba.

14. The method of claim 8, wherein the ingredients are combined in about equal proportions.

15. A product according to the method of claim 8.

16. A product according to the method of claim 13.

17. A product according to the method of claim 14.
